(19) United States
(12) Patent Application Publication
(10) Pub. No.: US 2022/0074760 A1
(43) Pub. Date: Mar. 10, 2022

(54) Method, apparatus and computer readable recording medium for providing user-customized geographic information and analysis information using universal map

(71) Applicant: Pluxity Co., Ltd., Seoul (KR)

(72) Inventors: Jaemin YOON, Seoul (KR); Sooyeon HAN, Seoul (KR); Suyeon CHO, Seoul (KR); Eunjeong JANG, Incheon (KR); Sunwoo KIM, Seoul (KR); Seunghyun YOON, Seoul (KR)

(21) Appl. No.: 17/417,535

(22) PCT Filed: Dec. 10, 2019
(86) PCT No.: PCT/KR2019/017382

§ 371 Date: Jun. 23, 2021

(30) Foreign Application Priority Data
Dec. 10, 2018 (KR) 10-2018-0168434
Sep. 11, 2019 (KR) 10-2019-0113038

(51) Int. Cl. G01C 21/36; G01C 21/00; G09B 21/00; A61B 5/16; A61B 5/372; A61B 5/024; A61B 5/11; G06F 21/31

(52) U.S. Cl. CPC G01C 21/3682; G01C 21/3889; G01C 21/3629; G09B 21/006; G06F 21/31; A61B 5/165; A61B 5/372; A61B 5/02438; A61B 5/1112; G09B 21/009

(57) ABSTRACT

Disclosed is a method for providing geographic information and analysis information for each user using a universal map, which includes: a map information receiving step of requesting authentication to a universal map server storing map information including one of a map image and guide information by transmitting user authentication information except socially disadvantaged type information, to receive map information from the universal map server; a guide information extraction step of extracting guide information, as guide information for providing the geographic information on the map image to the user terminal from the received map information, corresponding to the socially disadvantaged type information of the user among the user account information from a data model database; and a geographic information providing step of providing geographic information customized according to the user, by outputting geographic information reflecting attribute information of spatial objects to the user terminal based on the extracted guide information.

### BACKGROUND OF THE INVENTION

### 1. Field of the Invention

The present invention relates to a method for providing geographic information and analysis information for each user using a universal map perform authentication after transmitting user authentication information except for the user's socially disadvantaged type information to a universal map server, receive map information, and provide geographic information by extracting guide information corresponding to the socially disadvantaged type information of the user from a user terminal, so that personal information of the socially disadvantaged is prevented from being leaked to external network.

### 2. Description of the Related Art

The base of use of the geographic information services for the ordinary people has expanded to the degree of about 40 million users per month, due to the government's geographic information policy for the past 20 years.

However, it has been inappropriate for the socially disadvantaged, including the visually impaired, the hearing impaired, and the walking impaired, to use the services since most of general geographic information services are aimed at the ordinary people. Accordingly, the need for geographic information services for improving the living convenience and securing the mobility of the socially disadvantaged is emerging.

Meanwhile, as examples of the conventional geographic information service for the socially disadvantaged, Korean Patent Registration No. 10-0397480 (LOCATION INFORMATION SERVICE SYSTEM AND SERVICE METHOD FOR VISUALLY IMPAIRED) discloses a technology, as a geographic information service for the visually impaired, that outputs information on the current location and information to the next destination of a user in voice and Braille. Korean Utility Model Registration No. 20-0471699 (ROUTE GUIDANCE MAP FOR MOBILITY HANDICAPPED) discloses a technology that provides a user using a wheelchair and an auxiliary mobility apparatus with route blocks and on-the-spot photos of access-obstacle factors.

However, the described above related arts merely provide geographic information services for the specific socially disadvantaged. Particularly, since sensitive personal information such as a socially disadvantaged type may be leaked on external networks, the risk of damage due to the personal information leakage is very high.

### SUMMARY OF THE INVENTION

Accordingly, a primary object of the present invention is to perform authentication after transmitting user authentication information except for the user's socially disadvantaged type information to a universal map server, receive map information, and provide geographic information by extracting guide information corresponding to the socially disadvantaged type information of the user from a user terminal, so that customized geographic information is guided to the socially disadvantaged without leakage of sensitive personal information.

In addition, a secondary object of the present invention is that geographic information reflecting attribute information of particularly spatial objects is outputted when the geographic information is provided, so that geographical information with high utility is provided to the socially disadvantaged.

In order to achieve the technical problems, a method, which is implemented in a user terminal including at least one processor and at least one memory for storing instructions executable by the processor to provide geographic information and analysis information for each user using a universal map, includes: a map information receiving step in which authentication is requested to the universal map server storing map information including at least one of a map image and guide information for each socially disadvantaged type by transmitting user authentication information except for socially disadvantaged type information of a user among user account information requesting the authentication, and map information is received from the universal map server when the authentication is completed; a guide information extraction step of extracting guide information, as guide information for providing geographic information on the map image to the user terminal from the received map information, corresponding to the socially disadvantaged type information of the user among the user account information from a data model database storing data models for each socially disadvantaged type; and a geographic information providing step of providing geographic information customized according to the socially disadvantaged type information of the user, by outputting and providing geographic information reflecting attribute information of spatial objects existing on the map image to the user terminal based on the extracted guide information.

In the above-described geographical information providing step, it may be preferable that different surrounding POI information, as the geographic information, is visually outputted and provided according to the socially disadvantaged type of the user, on the map image activated in the user terminal.

In the above-described geographical information providing step, it may be preferable, through an output mechanism for providing geographic information according to the socially disadvantaged type of the user, that the geographic information is converted and outputted from text information into voice signals by using a text-to-speech (TTS) technology for the visually impaired, the geographic information is outputted as a sign language image for the hearing impaired, and the geographic information is outputted as a point of interest (POI) list for the walking impaired.

The above-described attribute information of the spatial object may preferably include accessibility information for support factor information and obstacle factor information, in which the support factor information and the obstacle factor information may be classified based on different classification criteria according to the socially disadvantaged type.

In the above-described geographical information providing step, a weight may be set to the accessibility information including at least one of the support factor information and the obstacle factor information according to the socially disadvantaged type information of the user and the attribute information of the spatial object, so as to determine a sequence of accessibility information to be outputted preferentially to the user terminal when the geographic information providing step is performed, and the above-mentioned accessibility information may be outputted to the user terminal in the form of an overlay with at least one screen displaying the accessibility information, in which the transparency of the screen may be controlled according to the weight set to the accessibility information, so that the screen set as having the highest weight may be outputted most opaquely.

In the above-described geographical information providing step, preview information on the accessibility information including at least one of the support factor information and the obstacle factor information is provided to the user terminal, geographic information for bypassing the accessibility information in which the bypass instruction is inputted may be re-outputted and provided when the user terminal determines that a bypass instruction is inputted with respect to one accessibility information included in the provided preview information, and a feature value of the accessibility information in which the bypass instruction is inputted may be analyzed, so as to provide geographic information automatically converted as geographic information having a route that bypasses accessibility information having a feature value greater than or equal to a preset similarity when following geographic information is provided.

In the above-described geographical information providing step, predicted use difficulty information on the geographic information may be further provided to the user terminal by predicting the use difficulty in which an item including at least one of the numbers and the density of support factor information and obstacle factor information included in the provided geographic information is reflected.

The above-described user terminal may collect information on the time the user stayed in a specific area and route information for a preset period of the user, as control information, by using stay time information for each location information on an area in which the user is located, and variation value information on the location information, which are measured from a sensor capable of collecting geographic information system (GIS) and a timer that are mounted on the user terminal, and the universal map server may be configured such that preferred stay place information derived based on stay time information for one area among the control information collected by a plurality of user terminals may be collected according to the socially disadvantaged type, so that preferred staying place information for each socially disadvantaged type is generated and stored in a database, and preferred route information derived by the matching rate of route information during a preset period is collected for each socially disadvantaged type among the location information collected by the user terminals, so that preferred route information for each socially disadvantaged type is generated and stored in the database.

The above-mentioned universal map server may perform commercial district analysis by extracting floating population number and stay time information for a point of interest (POI) in one area from the control information collected by the terminals, and classify a commercial district grade for each POI according to the commercial district analysis result to store the classified commercial district grade in the database, so that the commercial district grade for each POI may be visually outputted and provided on the geographic information provided to the user terminal when the geographic information providing step is performed.

The above-described user terminal, among photographed images of the user's field of view (FOV) photographed by a photographing device of the user terminal, may collect a first image, which is a photographed image, as control information, in which the time identically maintaining objects included in the FOV exceeds a preset first threshold time, and the universal map server may identify an object included in the first images collected from the user terminals through a comparison process with previously stored images of facilities to derive facility information corresponding to the objects, generate preferred facility information with respect to the facility information within a preset first threshold order in the order of the number of derived times among the derived facility information, and classify the generated preferred facility information based on the socially disadvantaged type, so that preferred facility information for each socially disadvantaged type may be generated, and the generated preferred facility information may be stored in the database.

The above-described user terminal serving as a user-wearable device may interwork with an electroencephalogram (EEG) analyzer capable of measuring the user's electroencephalogram to collect EEG information according to the user's location and time point in conjunction with a sensor capable of collecting geographic information system (GIS) and a timer that are mounted on the user terminal, and the universal map server may generate emotional information for each situation including information about the user's emotions at the user's location and time point by using the user's location, time point, and EEG information collected from the user terminal, and generate preference information about facilities for each socially disadvantaged type from users' situational emotional information collected from a plurality of user terminals, and store the generated preference information in the database.

The above-described user terminal serving as a user-wearable device may interwork with a heart rate sensor capable of measuring the user's heart rates and collect heart rate information according to the user's location and time point in conjunction with a sensor capable of collecting geographic information system (GIS) and a timer that are mounted on the user terminal, the universal map server may control the user's heart rate information at the user's location and time point by using the user's location, time point, and heart rate information collected from the user terminal, and classify the degree of urgency of the user according to a measured heart rate value when the user's heart rate information is determined as being out of the normal heart rate range, thereby transmitting an emergency status notification of the user according to the classified degree of urgency to a terminal of a control agency.

Meanwhile, the device, which is implemented by a user terminal including at least one processor and at least one memory for storing instructions executable by the processor to provide geographic information and analysis information for each user using a universal map, includes: a map information receiving unit configured to request authentication to the universal map server storing map information including at least one of a map image and guide information for each socially disadvantaged type by transmitting user authentication information except for socially disadvantaged type information of a user among user account information requesting the authentication, and receive map information from the universal map server when the authentication is completed; a guide information extraction unit for extracting guide information, as guide information for providing geographic information on the map image to the user terminal from the received map information, corresponding to the socially disadvantaged type information of the user among the user account information from a data model database storing data models for each socially disadvantaged type; and a geographic information providing unit for providing geographic information customized according to the socially disadvantaged type information of the user, by outputting and providing geographic information reflecting attribute information of spatial objects existing on the map image to the user terminal based on the extracted guide information.

On the other hand, according to the present invention provides a computer-readable recording medium storing instructions for allowing a computing device to perform the following steps including: a map information receiving step in which authentication is requested to a universal map server storing map information including at least one of a map image and guide information for each socially disadvantaged type by transmitting user authentication information except for socially disadvantaged type information of a user among user account information requesting the authentication, and map information is received from the universal map server when the authentication is completed; a guide information extraction step of extracting guide information, as guide information for providing geographic information on the map image to a user terminal from the received map information, corresponding to the socially disadvantaged type information of the user among the user account information from a data model database storing data models for each socially disadvantaged type; and a geographic information providing step of providing geographic information customized according to the socially disadvantaged type information of the user by outputting and providing geographic information reflecting attribute information of spatial objects existing on the map image to the user terminal based on the extracted guide information.

According to one embodiment of the present invention, the map information is received while providing only the authentication information except for the socially disadvantaged type information of the user terminal to the universal map server, so that personal information specifying the user can be prevented from being leaked to the network.

In addition, according to one embodiment of the present invention, a universal map, which stores map information including at least one of a map image and guide information for each socially disadvantaged type, is received, and only the guide information according to the socially disadvantaged type of the user is extracted from the user terminal and outputted to an output mechanism of the user terminal, geographic information, which is mainly focused on attribute information of spatial objects including the support factor information and the obstacle factor information, can be provided as geographic information according to the socially disadvantaged type of the user.

In addition, according to one embodiment of the present invention, the output mechanism for providing the geographic information is also customized according to the socially disadvantaged type to which the user belongs, so that desired geographic information can be easily obtained without any particular difficulty.

In addition, according to one embodiment of the present invention, the weight of accessibility information including at least one of support factor information and obstacle factor information is set according to the socially disadvantaged type information and the attribute information of the spatial object, and the sequence to be preferentially outputted to the user terminal is determined according to the set weight, thereby ensuring the simplicity of an output system of the geographic information, so that the convenience in use can be maximized.

In addition, according to one embodiment of the present invention, a feature value of the accessibility information in which the bypass instruction is inputted is analyzed among the provided geographic information to provide geographic information excluding accessibility information having a feature value greater than or equal to a threshold similarity when following geographic information is provided, so that a more enhanced customized geographic information service can be provided according to using patterns of the user.

### BRIEF DESCRIPTION OF THE DRAWINGS

FIG. 1 shows a flow chart of a method for providing geographic information and analysis information for each user using a universal map according to one embodiment of the present invention.

FIG. 2 shows a conceptual diagram of the method for providing geographic information and analysis information for each user using a universal map according to one embodiment of the present invention.

FIG. 3 shows examples in which geographic information indicating different surrounding POI information is provided according to a socially disadvantaged type according to one embodiment of the present invention.

FIG. 4 shows examples in which different geographical information output mechanisms are used for socially disadvantaged types according to one embodiment of the present invention.

FIG. 5 shows examples of classifying obstacle factors and support factors according to the socially disadvantaged type according to one embodiment of the present invention.

FIG. 6 shows one example in which an output sequence is controlled according to weights set to support factor information and obstacle factor information according to one embodiment of the present invention.

FIG. 7 shows one example in which the degree of difficulty in using geographic information is provided to a user terminal according to one embodiment of the present invention.

FIG. 8 shows one example in which different accessibility information is provided according to the socially disadvantaged type of the user when the user arrives at a predetermined destination according to one embodiment of the present invention.

FIG. 9 shows one example for explaining a flow in which control information is managed according to one embodiment of the present invention.

FIGS. 10 and 11 show examples in which images photographed by a user terminal are utilized according to one embodiment of the present invention.

FIG. 12 shows one example in which commercial district analysis is performed using floating population numbers and stay times of socially disadvantaged users according to one embodiment of the present invention.

FIG. 13 shows one example for explaining a flow in which emotional information for each situation is utilized according to one embodiment of the present invention.

FIG. 14 shows one example in which the heart rates of socially disadvantaged users are monitored according to one embodiment of the present invention.

FIG. 15 shows a configuration diagram of a device for providing geographic information and analysis information for each user using a universal map according to one embodiment of the present invention.

FIG. 16 shows one example of an internal configuration of a computing device according to one embodiment of the present invention.

### DETAILED DESCRIPTION OF THE INVENTION

Hereinafter, various embodiments and/or aspects will be described with reference to the drawings. In the following description, numerous specific details are set forth in order to provide a thorough understanding of one or more aspects for the purpose of explanation. However, it will also be appreciated by a person having ordinary skill in the art that such aspect(s) may be carried out without the specific details. The following description and accompanying drawings will be set forth in detail for specific illustrative aspects among one or more aspects. However, the aspects are merely illustrative and some of various ways among principles of the various aspects may be employed, and the descriptions set forth herein are intended to include all the various aspects and equivalents thereof.

The terms “embodiment”, “example”, “aspect” or the like used herein may not be construed in that an aspect or design set forth herein is preferable or advantageous than other aspects or designs.

In addition, the terms “include” and/or “comprise” specify the presence of the corresponding feature and/or element, but do not preclude the possibility of the presence or addition of one or more other features, elements or combinations thereof.

In addition, the terms including an ordinal number such as first and second may be used to describe various elements, however, the elements are not limited by the terms. The terms are used only for the purpose of distinguishing one element from another element. For example, the first element may be referred to as the second element without departing from the scope of the present invention, and similarly, the second element may also be referred to as the first element. The term “and/or” includes any one of a plurality of related listed items or a combination thereof.

Further, in embodiments of the present invention, unless defined otherwise, all terms used herein including technical or scientific terms have the same meaning as commonly understood by those having ordinary skill in the art. Terms such as those defined in generally used dictionaries will be interpreted to have the meaning consistent with the meaning in the context of the related art, and will not be interpreted as an ideal or excessively formal meaning unless expressly defined in an embodiment of the present invention.

The present invention relates to a method for providing geographic information and analysis information for each user using a universal map. A primary object of the present invention is to perform authentication after transmitting user authentication information except for the user's socially disadvantaged type information to a universal map server, receive map information, and provide geographic information by extracting guide information corresponding to the socially disadvantaged type information of the user from a user terminal, so that customized geographic information is guided without leakage of sensitive personal information. A secondary object of the present invention is that geographic information reflecting attribute information of spatial objects is outputted, so that geographical information with high utility is provided to the socially disadvantaged.

In order to achieve the above objects, hereinafter, the present invention will be described in more detail with reference to the drawings.

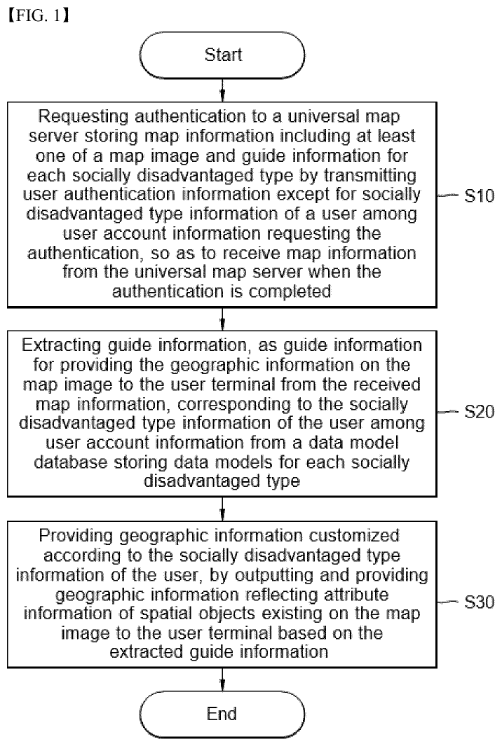

Meanwhile, FIG. 1 shows a flow chart of a method for providing geographic information and analysis information for each user using a universal map according to one embodiment of the present invention.

Referring to FIG. 1, first, a map information receiving step S10 may be performed, in which authentication is requested to a universal map server including at least one of a map image and guide information for each socially disadvantaged type by transmitting user authentication information except for socially disadvantaged type information of a user among user account information requesting the authentication, and map information is received from the universal map server when the authentication is completed.

In regard to the above-described universal map server, it may be understood that the above-described map image is an image composed of points, edges, and planes to indicate a space and a region, and it may be understood that an image object that easily represents usual geographic information.

In addition, the above-described guide information for each socially disadvantaged type may be understood that the socially disadvantaged type is classified, for example, into visually impaired, hearing impaired, and walking impaired with a wheelchair or infant, and output information of the guide information output mechanism is set differently according to each socially disadvantaged type.

On the other hand, in step S10, the user terminal may request the authentication and receive the universal map information by transmitting user authentication information except for the socially disadvantaged type information of the user to the universal map server.

In the above-described step S10, it may be understood that the user authentication information except for the socially disadvantaged type information of the user is configured to include, for example, at least one of account information (ID) of the user terminal and location information inputted from the user terminal.

In other words, in step S10, it may be understood that the guide information for all socially disadvantaged types is received to the user terminal through the user authentication information. In the above-described step S10, the user's socially disadvantaged type information is not allowed to be provided to the universal map server, so that the occurrence of leaking user's sensitive personal information to the outside can be fundamentally blocked.

Meanwhile, after step S10 is performed, a guide information extraction step S20 may be performed, in which guide information corresponding to the socially disadvantaged type information of the user among the user account information, as guide information for providing geographic information on the map image to the user terminal from the map information received in step S10, is extracted from a data model database storing data models for each socially disadvantaged type.

The above-described data model database may be understood as a mechanism for storing and managing each guide information for at least one socially disadvantaged type, that is, for the walking impaired, the visually impaired and the hearing impaired.

Specifically, it is desirable to understand that the data model for each socially disadvantaged type stored in the above-described data model database is a concept that stipulates a geographic information extraction standard for each socially disadvantaged type, an accessibility information extraction standard including at least one of obstacle factor information and support factor information, and a POI information display standard. The detailed description thereof will be provided later.

In other words, it may be understood that, because the map information received in step S10 includes guide information for all socially disadvantaged type information as mentioned above, the process of extracting necessary guide information according to the socially disadvantaged type of the user is performed in step S20 in order to prevent the socially disadvantaged type information, which is sensitive personal information of the user, from being leaked.

When the socially disadvantaged type of the user is, as an example, the walking impaired, guide information corresponding to the walking impaired may be extracted in step S20.

In other words, when step S20 is performed, the geographic information suitable for the socially disadvantaged type of the user may be provided from the user terminal.

Meanwhile, after step S20 is performed, a geographic information providing step S30 may be performed, in which geographic information customized according to the socially disadvantaged type information of the user is provided by outputting and providing geographic information reflecting attribute information of spatial objects existing on the map image to the user terminal based on the extracted guide information.

In the above-described step S30, the geographic information may be provided to the output mechanism of the user terminal, in which different surrounding POI information may be visually outputted according to the socially disadvantaged type of the user on a map image activated in the output mechanism of the user terminal.

More specifically, surrounding POI information is displayed based on the socially disadvantaged type of the user even when geographic information of the same area is provided, in which surrounding POI information on places installed with wheelchair chargers may be displayed on a map image in a terminal of a user requiring a wheelchair as a walking impaired; surrounding POI information on places where a nursing room is located may be displayed on a map image in a terminal of a user corresponding to a family with an infant; and surrounding POI information on places equipped with warning lights may be displayed on a map image for the hearing impaired.

Meanwhile, the visualization mechanism of the above-described surrounding POI information may be visualized by a graphic symbol including at least a pictogram, and the present invention is not limited thereto.

On the other hand, according to the present invention, when it is determined that there is a selection input for the surrounding POI information provided according to the socially disadvantaged type in the user terminal, detailed information including at least one of traffic information, operating hours, contact information, and tourism information may be further provided for the selectively inputted surrounding POI information.

The above-described detailed information on the surrounding POI information may also be provided in different ways for socially disadvantaged types of users. Accordingly, the hearing impaired may be provided with the detailed information on the surrounding POI information as texts and sign language images, and the visually impaired may be provided with voice signals based on a TTS technology.

In other words, according to the above-described embodiment of the present invention, geographic information providing mechanism with maximized recognition convenience can be provided to the user belonging to the socially disadvantaged class having many physical limitations.

In addition, the attribute information of the above-described spatial object may include accessibility information on support factor information and obstacle factor information with respect to the socially disadvantaged user. Specifically, the above-described support factor information and obstacle factor information may be classified based on different classification criteria depending on the socially disadvantaged type.

In other words, different classification criteria may be set even for attribute information of the same spatial object with respect to each socially disadvantaged type.

As an example, when it is assumed that there are walking impaired A and visually impaired B, and when a road boundary stone is present as attribute information of the spatial object, the road boundary stone may serve as the obstacle factor to the walking impaired A, but may serve as the supporting factor to the visually impaired B for identifying the road from the sidewalk.

As another example, when it is assumed that there are walking impaired A and visually impaired B, and when a pedestrian overpass exists as attribute information of a spatial object, the pedestrian overpass may serve as the obstacle factor of interrupting the moving of the walking impaired A side, but may serve as a route support factor safer than a crosswalk on the visually impaired B side.

In other words, according to the present invention as shown in the above embodiments, it is desirable to understand that the classification criteria may be differently applied to the support factor and the obstacle factor for each socially disadvantaged type.

Meanwhile, in the above-described step S30, the geographic information may be provided by considering characteristics on the attribute information of the spatial objects, so as to output and provide the geographic information according to the socially disadvantaged type of the user to the user terminal.

Specifically, when the socially disadvantaged type of the user is the visually impaired, the geographic information may be provided by converting text information into voice signals using a text-to-speech (TTS) technology.

It may be understood that the above-described TTS technology is a technology that converts text information or symbols into human voice, and the above-described TTS may include a natural language processing technology configured to build pronunciation data for all phonemes of a language, generate a continuous voice by connecting the pronunciation data, and synthesize natural voices by adjusting the size, length, and pitch of the voice.

In other words, it may be understood that, when the fact that the user cannot see the map image provided to the output mechanism (such as a display) of the user terminal due to the visual impairment is considered, the text inserted into the object on the map image for route guidance is converted into speech, so that the guide of the geographic information may be provided using an output mechanism (such as a speaker) of the user terminal.

As another embodiment, when the socially disadvantaged type of the user is the hearing impaired, the geographic information may be outputted as a sign language image.

It may be understood that the above-described sign language image is a communication scheme that delivers meanings by visual ways including hand gestures, body gestures, and facial expressions. In other words, according to the present invention, the geographic information is guided through the sign language images to the hearing impaired side having difficulty in hearing sounds, so that the delivery power of the provided geographic information may be further maximized.

As another embodiment, when the socially disadvantaged type of the user is the walking impaired using a wheelchair or stroller, the geographic information may be outputted as a point of interest (POI) list.

It may be understood that the above-described POI provides geographic information based on location information on major facilities around roads, so that the geographic information can be guided in an easier form.

In other words, it may be understood that the output mechanism corresponding to the socially disadvantaged type is selected in the above-described step S30, so that the geographic information may be provided.

Meanwhile, upon providing the geographic information that reflects the above-mentioned attribute information of the spatial object, it may be understood that geographic information describing the installation form of the spatial object in detail is provided in order to improve the walking mobility of the socially disadvantaged.

As an example, in the attribute information of the spatial object, information on the presence of a ramp, the presence of a step, and the presence of a stair guardrail may be provided with respect to a main entrance access road; information on the width of an opening and the opening/closing type of the door (for example, a rotating door and an opening/closing direction) may be provided with respect to information about an entrance door; and information on the presence of a toilet for the impaired, the installation of a diaper exchange table, and the presence of gender-specific signs may be provided in the geographic information with respect to information about a toilet.

More preferably, according to the present invention, a weight is set to the accessibility information including at least one of support factor information and obstacle factor information based on the socially disadvantaged type information of the user and the attribute information of the spatial object, so that the sequence of the accessibility information to be preferentially outputted to the user terminal may be determined when the geographic information is provided.

Meanwhile, it may be understood that the above-described weight may be an option selected and inputted by the user terminal. In addition, it may be understood that, when the user sets a higher weight on the obstacle factor information than the support factor information, information on the obstacle factor may be preferentially outputted to the user terminal upon providing the geographic information.

More specifically according to the present invention, when the above-described accessibility information is provided to the user terminal, a screen displaying the accessibility information may be outputted in the form of at least one overlay.

In other words, it may be understood that the accessibility information set with the highest weight is outputted as a top overlay, and the accessibility information is displayed on a lower overlay as a weight set value is lower.

Meanwhile, according to the present invention, transparency may be controlled according to the weight set value, in addition to the screen output having the overlay form.

In other words, the screen set with the highest weight is configured to output the most opaque screen, so that the delivery of geographic information can be simplified and maximized.

On the other hand, in the above-described step S30 according to another embodiment of the present invention, preview information on the accessibility information including at least one of support factor information and obstacle factor information may be provided to the user terminal.

Specifically, it may be understood that the above-described preview information refers to information that guides information on supporting factors and obstacle factors existing in a section of space or route in advance when geographic information including at least any one of information on a predetermined space and route information on a predetermined destination is provided in the user terminal.

In other words, it may be understood that, as an example, when a request for geographic information about a route to cultural center A from the current location is received from the user terminal, a function of providing support factor information and obstacle factor information on the route to the user terminal in advance is performed.

Meanwhile, according to the present invention, in addition to simply providing the above information, when the user terminal determines that a bypass instruction is inputted with respect to one accessibility information included in the preview information provided to the user terminal, geographic information, which is configured to bypass the accessibility information in which the bypass instruction is inputted, may be re-outputted and provided.

In other words, when it is determined that there is a factor unfavorable to the socially disadvantaged user on the route and there is a factor that is difficult to use, a route for enabling the user to bypass the unfavorable route is provided rather than use the unfavorable route against the user's will, so that the user who is the socially disadvantaged can be protected from risky situations, and an accident can be prevented.

Meanwhile, in the above-described embodiment according to the present invention, a feature value of the accessibility information in which the bypass instruction is inputted is analyzed, so that geographic information, which is obtained by automatically converting accessibility information having a feature value the same as or greater than or equal to a preset similarity into geographic information having a bypass route, may be provided when following geographic information is provided.

According to the present invention, as one example, when the factor for which the bypass instruction is inputted from the user terminal is a revolving door, and when geographic information to be performed next is provided, geographic information for enabling the user bypass the revolving door is provided, so that the enhanced user-customized geographic information may be provided.

In other words, due to the above configuration according to the present invention, the customized geographic information that reflects the socially disadvantaged information and propensity of the user can be provided, and accordingly, satisfaction on the geographic information providing service can be maximized.

Meanwhile, in another embodiment of step S30 according to the present invention, predicted use difficulty information on the geographic information may be further provided to the user terminal by predicting the use difficulty in which an item including at least one of the numbers and the density of support factor information and obstacle factor information included in the geographic information provided to the user terminal is reflected,

It may be preferable to understand that the above-described use difficulty may be determined by calculating the number of accessibility information existing in a predetermined place or a predetermined route, and considering the ratio and density of the support factors and the obstacle factors.

As a simple example, when the obstacle factors are more than the support factors on a predetermined route, it may be determined as being significantly difficult to use the route due to the high use difficulty. On the contrary, when the support factors are more than the obstacle factors, it may be determined as being a route to be easily used due to the low use difficulty.

In other words, according to the present invention, the information on the use difficulty is further provided as the geographic information, thereby enabling the socially disadvantaged user to select a route based on the use difficulty, so that the satisfaction in using the service can be maximized.

On the other hand, as another embodiment of step S30 of the present invention, upon performing step S30 according to the present invention, it may be preferable to determine whether the user deviates from the route by comparing the geographic information with the user's real-time location received from a GPS module equipped in the user terminal.

Meanwhile, it may be understood that the determination on whether the user deviates from the route is performed when geographic information for guiding a route to a predetermined destination is provided to the user. At this point, according to the present invention, information about both cases when the user deviates the route and the user does not deviate the route may be outputted as a differentiated notification to the user terminal.

As an example, when it is assumed that the user is being provided with geographic information about the destination, and when the user terminal determines that the user is moving to the destination on the proper route without deviating from the route, the user terminal may output a short and weak vibration for about 1 second in every preset period or route guidance section. On the contrary when the user terminal determines that the user has deviated from the route, the user terminal may continuously output a long and strong vibration for about 3 seconds.

In other words, the above configuration may provide a real-time feedback on the deviation of route to the user in the form of tactile feedback by using the user terminal, so that the route deviation can be easily recognized. Accordingly, the geographic information can be guided more smoothly.

Meanwhile, in a more exemplary embodiment of the present invention, the user terminal described in the present invention is equipped with a sensor and timer that may collect a geographic information system (GIS), and the information is collected and stored in the database, so that additional information meaningful to the geographic information provided to the socially disadvantaged user may be provided.

As a specific example, the user terminal used in the present invention may collect information on the time the user stayed in a specific area and route information for a preset period of the user, as control information, by using stay time information for each location information on an area in which the user is located, and variation value information on the location information.

At this point, when stay time information for each location information is collected as the control information, the time the user stays in a specific location may be checked to determine whether the user has a preference for scenery and facilities provided in the corresponding location. When time-based variation value information of the location information is used, moving paths for a preset period of time (such as one day) may be analyzed.

In other words, the universal map server may collect the control information from user terminals, and preferred stay place information derived based on stay time information for one area among the control information collected by a plurality of user terminals may be collected according to the socially disadvantaged type, so that preferred staying place information for each socially disadvantaged type may be generated and stored in the database,

When routes traveled by user terminals for a predetermined period of time overlaps each other, and when the overlapping routes are collected according to each socially disadvantaged type, routes overlapping for each socially disadvantaged type may be derived, and the above routes may be defined as routes which are easy to move for a specific socially disadvantaged type or routes through which users want to move, so that the routes may be defined as preferred routes for each socially disadvantaged type.

In other words, accordingly, the above-described preferred place of stay and the preferred route can solve the problem of providing meaningless, that is, useless information when simple information is provided, and the staying places actually used by users may be provided according to the socially disadvantaged type, so that more meaningful geographic information can be provided to the socially disadvantaged types.

Meanwhile, in another embodiment of the present invention, information on floating population number and stay time of the socially disadvantaged users for POI in one area may be extracted using the collected control information, so that a function of a POI-specific commercial district analysis may be performed.

Specifically, according to the above-described POI-specific commercial district analysis, the commercial district may be analyzed as better when there are more floating populations of the user terminal with respect to the POI and the stay time is longer for a predetermined period, and the commercial district may be analyzed as worse when the floating population is less and the stay time is shorter.

In regard to the above-described commercial district analysis, the result of commercial district analysis analyzed based on the information about floating populations and stay time may be classified into a preset grade, so that the commercial district analysis result information may be visually provided on the map provided to the user terminal.

In the commercial district analysis result information, it may signify that the more it is analyzed as an excellent commercial district, the more convenient facilities are available for the socially disadvantaged users, and accordingly, meaningful geographic information can be provided to the socially disadvantaged users.

Meanwhile, as another embodiment of the present invention, when the user terminal is installed with a camera as a device capable of photographing at least field of view (FOV) of the user, for example, capable of photographing an image in the same direction as the user's view, the user terminal may collect a first image, which is a photographed image corresponding to a time for which an object included in the FOV is identically maintained exceeds a preset first threshold time, as control information, among the photographed images of the user's FOV photographed by the photographing device of the user terminal.

In order to perform the above-described function, it may be understood that the user terminal includes a process for deriving boundary values by analyzing pixels or the like from the image, and a program/application or function for identifying objects by using the process.

In addition, the user terminal may continuously/periodically photograph images. At this point, when each object is identified from the photographed images, and there is an image in which the identified object exceeds a predetermined amount of time, that is, the first threshold time (for example, 5 minutes), the image may be defined as a first image and collected as control information.

The first image may be all images photographed while exceeding the first threshold time, or a photographed image corresponding to a specific time (first, middle, or last point), or may be defined as an image of only the corresponding object, that is, an image obtained by separating only an image of the object from the photographed image.

Meanwhile, the universal map server may collect the control information from each user terminal, and identify objects included in the first images collected from the multiple user terminals through a comparison process with previously stored facility images, thereby deriving facility information corresponding to the objects.

To this end, images of objects corresponding to facilities may be matched for the facilities and stored in the database, in which the facilities refer to all large or small devices for socially disadvantaged types, such as specific facilities and equipment applicable for the socially disadvantaged users.

In addition, it may be desirably understood that the universal map server may generate preferred facility information with respect to the facility information within a preset first threshold order (for example, five facilities) in the order of the number of derived times among the derived facility information, and then classify the generated preferred facility information according to the socially disadvantaged class, so that preferred facility information for each socially disadvantaged class may be generated and stored in the database.

On the other hand, in another embodiment of the present invention, the user terminal as a device wearable by the user may interwork with an electroencephalogram (EEG) analyzer capable of measuring the user's electroencephalogram or a heart rate measuring device, and may collect EEG information or heart rate information according to the user's location and time point in conjunction with a sensor capable of collecting GIS and a timer that are mounted on the user terminal.

It may be understood that the above-mentioned EEG information generates emotional information for each situation including information about the user's emotions at the user's location and time point to generate preference information about facilities for each socially disadvantaged type and stores the generated preference information in the database. In addition, it may be understood that the above-mentioned heart rate information is used as a means for controlling the user's urgency.

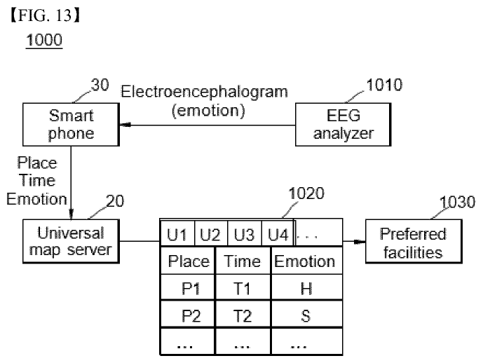

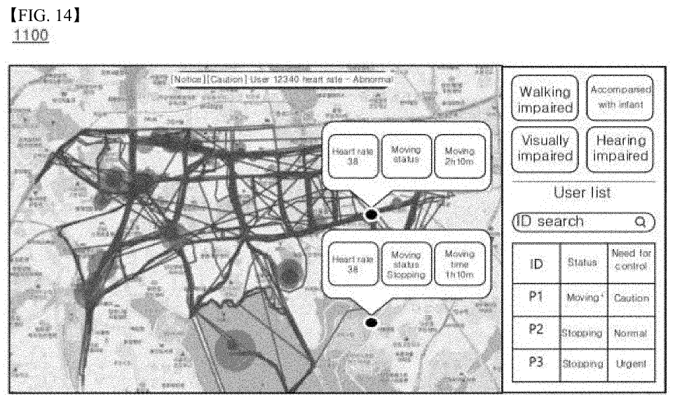

Meanwhile, more detailed description for the above-mentioned collection of EEG information and heart rate information will be provided in more detail with reference to FIGS. 13 and 14 described later.

Comprehensively, according to one embodiment of the present invention, the map information is received while providing only the authentication information except for the socially disadvantaged type information of the user terminal to the universal map server, so that personal information specifying the user can be prevented from being leaked to the network.

In addition, according to one embodiment of the present invention, a universal map, which stores map information including at least one of a map image and guide information for each socially disadvantaged type, is received, and only the guide information according to the socially disadvantaged type of the user is extracted from the user terminal and outputted to an output mechanism of the user terminal, so that geographic information, which is mainly focused on attribute information of spatial objects including the support factor information and the obstacle factor information, can be provided as geographic information according to the socially disadvantaged type of the user.

In addition, according to one embodiment of the present invention, the output mechanism for providing the geographic information is also customized according to the socially disadvantaged type to which the user belongs, so that desired geographic information can be easily obtained without any particular difficulty.

In addition, according to one embodiment of the present invention, the weight of accessibility information including at least one of support factor information and obstacle factor information is set according to the socially disadvantaged type information and the attribute information of the spatial object and the sequence to be preferentially outputted to the user terminal is determined according to the set weight, thereby ensuring the simplicity of an output system of the geographic information, so that the convenience in use can be maximized.

In addition, according to one embodiment of the present invention, a feature value of the accessibility information in which the bypass instruction is inputted is analyzed among the provided geographic information to provide geographic information excluding accessibility information having a feature value greater than or equal to a threshold similarity when following geographic information is provided, so that a more enhanced customized geographic information service can be provided according to using patterns of the user.

Although the above embodiments have been described with reference to the limited embodiments and drawings, it will be understood by those skilled in the art that various changes and modifications may be made from the above-mentioned description.

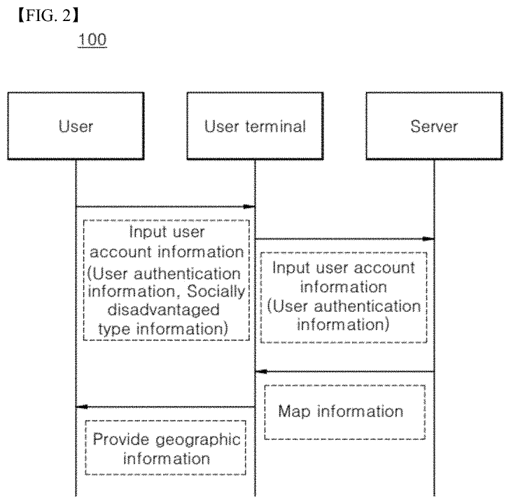

FIG. 2 shows a conceptual diagram of the method for providing geographic information and analysis information for each user using the universal map according to one embodiment of the present invention. In the following description, unnecessary descriptions for embodiments redundant with those of FIG. 1 will be omitted.

As shown in screen 100 of FIG. 2, the present invention may be largely described in terms of the user side, the user terminal side, and the universal map server side.

Specifically, the user may enter user account information in the user terminal, in which the user account information may include authentication information, such as an ID assigned to the user, and socially disadvantaged type information.

The user terminal in which the user account information is inputted transmits only user authentication information excluding the socially disadvantaged type information among the user's account information to the universal map server, so that the authentication may be performed.

In other words, according to the present invention, only the information necessary for user authentication among the user account information is transmitted to the universal map server, so that sensitive personal information including the socially disadvantaged type information of the user can be prevented from being leaked to external network.

Meanwhile, when the authentication is completed through the user authentication information, the universal map server transmits map information including a map image and guide information for each socially disadvantaged type to the user terminal, and the user terminal receiving the map information extracts guide information corresponding to the socially disadvantaged type of the user based on the socially disadvantaged type information stored in the user terminal to output the extracted guide information to the output mechanism, so that the geographic information may be provided to the user.

The above-described geographic information is geographic information according to the socially disadvantaged type of the user, and the geographic information, which is centered on attribute information of spatial objects including support factor information and obstacle factor information, may be provided, so that the user as the socially disadvantaged can easily obtain the desired geographic information without any difficulty.

Meanwhile, the above-described geographic information may also be used as a means for guiding the route when location information about start and destination is inputted in the user terminal, and the present invention is not limited thereto.

Although the above embodiments have been described with reference to the limited embodiments and drawings, it will be understood by those skilled in the art that various changes and modifications may be made from the above-mentioned description.

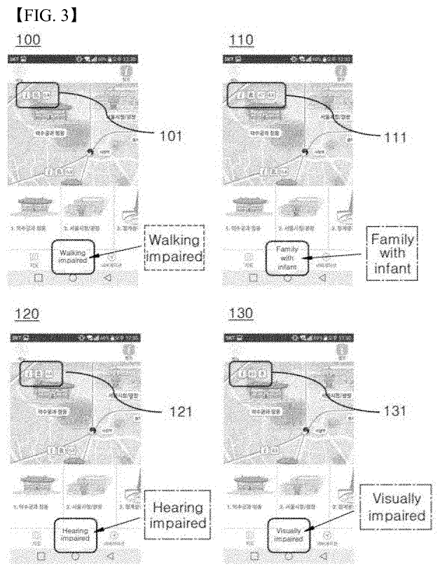

FIG. 3 shows an embodiment in which geographic information indicating different surrounding POI information is provided based on the socially disadvantaged type according to one embodiment of the present invention. In the following description, unnecessary descriptions for embodiments redundant with those of FIGS. 1 and 2 will be omitted.

It may be understood that screens 100 to 130 of FIG. 3 show map images outputted to provide geographic information to terminals of the walking impaired, a family companied with an infant, the hearing impaired, and visually impaired that move inconveniently, and all of the map images 100 to 130 are examples provided as the same area.

First, the screen 100 is a screen provided to the user terminal of the walking impaired. As indicated in reference numeral 101, POI information, such as information about an information center, information about a parking lot for the impaired, and information about a toilet for the impaired, is displayed by using pictograms as the surrounding POI information.

The screen 110 is a screen provided to the user terminal of the family companied with the infant. As indicated in reference numeral 111, POI information, such as information about an information center, information about a parking lot for the impaired, information about a toilet for the impaired, and information about a stroller rental site, is displayed by using pictograms as the surrounding POI information.

Screen 120 is a screen provided to the user terminal of the hearing impaired. As indicated in reference numeral 121, POI information, such as information about an information center, information about a parking lot for the impaired, and information about a toilet for the impaired, is displayed by using pictograms as the surrounding POI information.

The screen 130 is a screen provided to the user terminal of the visually impaired. As indicated in reference numeral 131, POI information, such as information about an information center, information about a toilet for the impaired, and information about a voice guide rental site, is displayed by using pictograms as the surrounding POI information.

Meanwhile, although not shown in reference numeral 130, a mode for color weakness may be set in the user terminal of the visually impaired, so the surrounding POI information may be outputted as a gray scaled pictogram having a relatively high density.

In other words, as described in the embodiments of screens 100 to 130 according to the present invention, different surrounding POI information, as geographic information, may be visually outputted according to the socially disadvantaged type of the user on a map image activated in the output mechanism of the user terminal. Accordingly, geographic information providing mechanism with maximized recognition convenience can be provided to the user belonging to the socially disadvantaged class having many physical limitations.

Meanwhile, in the embodiment of FIG. 3, the description is limited as simply displaying the surrounding POI information by using the pictograms, however, a message may be provided about a list of the surrounding POI by opening a pop-up window in one area of the output screen of the user terminal to which the map image is extracted, and the present invention is not limited thereto.

Although the above embodiments have been described with reference to the limited embodiments and drawings, it will be understood by those skilled in the art that various changes and modifications may be made from the above-mentioned description.

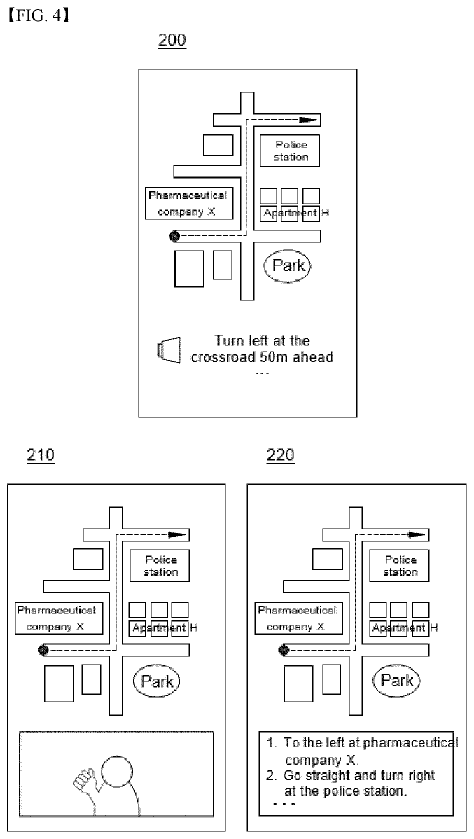

FIG. 4 shows examples in which different geographical information output mechanisms are used for socially disadvantaged types according to one embodiment of the present invention.

Likewise, in the following description, unnecessary descriptions for embodiments redundant with those of FIGS. 1 to 3 will be omitted.

Referring to FIG. 4, it is assumed that a route to a predetermined destination is guided as geographic information in the user terminal.

Screen 200 is an embodiment in which the geographic information is outputted to the visually impaired. The provision of geographic information may be outputted to the visually impaired through a speaker equipped in the user terminal by converting text information inserted into a spatial object into a voice, Accordingly, the geographic information may be more clearly delivered to the visually impaired who cannot see the map image.

Meanwhile, screen 210 is an embodiment in which the geographic information is outputted to the hearing impaired. A sign language video may be outputted, together with the map image, on a display of the user terminal of the hearing impaired, Accordingly, the geographic information may be more clearly delivered to the hearing impaired who cannot hear sound.

On the other hand, screen 220 is an embodiment in which the geographic information is outputted to the walking impaired. The POI list may be provided, together with the map image, on a display of the user terminal of the walking impaired. Meanwhile, in regard to the POI list, it may be desirable to provide a POI list having routes that the walking impaired may use. It may be understood that, as an example, the POI list is provided for areas in which wheelchairs are accessible, and area in which doors have widths accessible for wheelchairs and strollers

In other words, according to the present invention, geographic information, which is mainly focused on attribute information of spatial objects including the support factor information and the obstacle factor information, can be provided as geographic information according to the socially disadvantaged type of the user, so that the socially disadvantaged user can easily obtain the desired geographic information without any difficulty. Although the above embodiments have been described with reference to the limited embodiments and drawings, it will be understood by those skilled in the art that various changes and modifications may be made from the above-mentioned description.

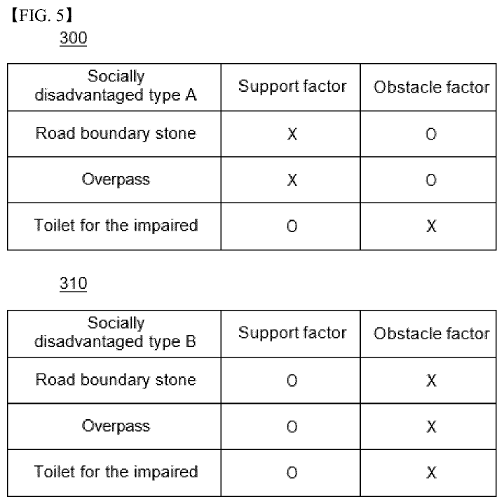

FIG. 5 shows examples of classifying obstacle factors and support factors according to the socially disadvantaged type according to one embodiment of the present invention. In the following description, unnecessary descriptions for embodiments redundant with those of FIGS. 1 to 4 will be omitted.

As mentioned above, upon providing the geographic information to the user terminal according to the present invention, the geographic information corresponding to the socially disadvantaged type of the user is outputted and provided as geographic information in which attribute information of spatial objects existing on the map image is reflected.

The above-described attribute information of the spatial object may include accessibility information on support factor information and obstacle factor information. Thus, according to the present invention, the geographic information including information on support factors and obstacle factors is provided to the user terminal.

Meanwhile, the support factor information and the obstacle factor information may have different classification criteria depending on the socially disadvantaged type, and embodiments thereof are shown in reference numerals 300 and 310 of FIG. 5.

At this point, it may be understood that reference numeral 300 is a classification table of support factor information and obstacle factor information for socially disadvantaged type A (walking impaired), and it may be understood that reference numeral 310 is a classification table of support factor information and obstacle factor information for socially disadvantaged type B (visually impaired).

For the socially disadvantaged type A, a road boundary stone and a pedestrian overpass may act as obstacle factors. However, for the socially disadvantaged type B, the road boundary stone and the pedestrian overpass may act as support factors.

In other words, according to the present invention, the geographic information may be provided by considering that even attribute information of the same spatial object may serve as a support factor or as an obstacle according to the socially disadvantaged type, so that the geographic information provision service provided to the socially disadvantaged can be more systematically implemented.

Although the above embodiments have been described with reference to the limited embodiments and drawings, it will be understood by those skilled in the art that various changes and modifications may be made from the above-mentioned description.

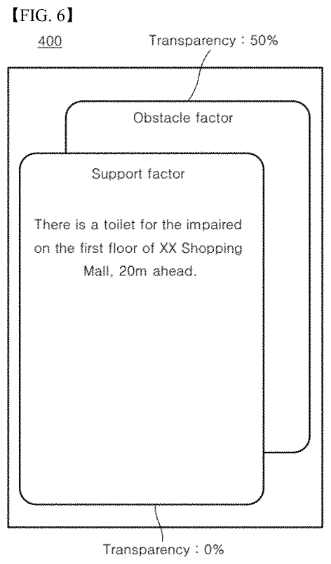

FIG. 6 shows one example in which an output sequence is controlled according to weights set to support factor information and obstacle factor information according to one embodiment of the present invention.

In the following description, unnecessary descriptions for embodiments redundant with those of FIGS. 1 to 5 will be omitted.

Referring to screen 400 of FIG. 6, screen 400 is an embodiment when a higher weight is set to the support factor information compared to the obstacle factor information, and accordingly, the support factor information is exposed at the top.

According to the present invention as mentioned above, the accessibility information may be displayed in the form of an overlay according to the weight to control the transparency according to the weight. Accordingly, the transparency may be set as 0% in the support factor information set with high weight so as to be outputted in an opaque form, and the transparency may be set as 50% in the obstacle factor information having a relatively low weight so as to be outputted to a screen with transmittance.

In other words, according to the present invention, a mechanism for improving the delivery of geographical information is provided while having a simple screen configuration, so that the convenience in use can be maximized in terms of the socially disadvantaged users.

Although the above embodiments have been described with reference to the limited embodiments and drawings, it will be understood by those skilled in the art that various changes and modifications may be made from the above-mentioned description.

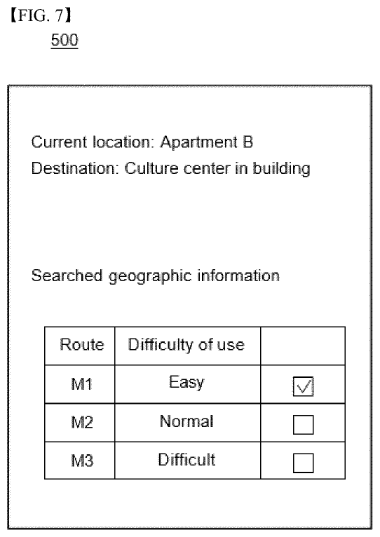

FIG. 7 shows one example in which the degree of difficulty in using the geographic information is provided to the user terminal according to one embodiment of the present invention. In the following description, unnecessary descriptions for embodiments redundant with those of FIGS. 1 to 6 will be omitted.

Referring to screen 500 of FIG. 7 according to the present invention, predicted use difficulty information on the geographic information may be further provided to the user terminal by predicting the use difficulty in which an item including at least one of the numbers and the density of support factor information and obstacle factor information included in the provided geographic information is reflected.

The above-described use difficulty may be calculated by calculating the number of accessibility information existing in a predetermined place or a predetermined route, and analyzing the number, ratio, and density of support factors and obstacle factors.

More specifically, the use difficulty may be calculated by analyzing the number and density of supporting factors and obstacle factors with respect to a distance of the entire route.

In other words, it may be understood that, in screen 500, when at least one geographic information about the route from the user's current location to the cultural center in building A is provided, geographic information for each difficulty level may be provided. Although screen 500 shows only the information about the use difficulty, required time information and distance information may be further included.

Accordingly, it may be understood that the user may be provided with a guide for any one of the geographic information provided by comprehensively considering information on the required time, moving distance, and difficulty. Accordingly, the socially disadvantaged user may be allowed to select a route based on the use difficulty, so that the satisfaction in using the service can be maximized.

Meanwhile, the embodiment of screen 500 of FIG. 7 has described the example in which the use difficulty is limited to a three-level system of high, medium, and low. However, it may be desirably understood that the use difficulty for each grade may be further subdivided, and the use difficulty may be calculated as a quantified score by assigning a score to each attribute information of the spatial object.

Although the above embodiments have been described with reference to the limited embodiments and drawings, it will be understood by those skilled in the art that various changes and modifications may be made from the above-mentioned description.

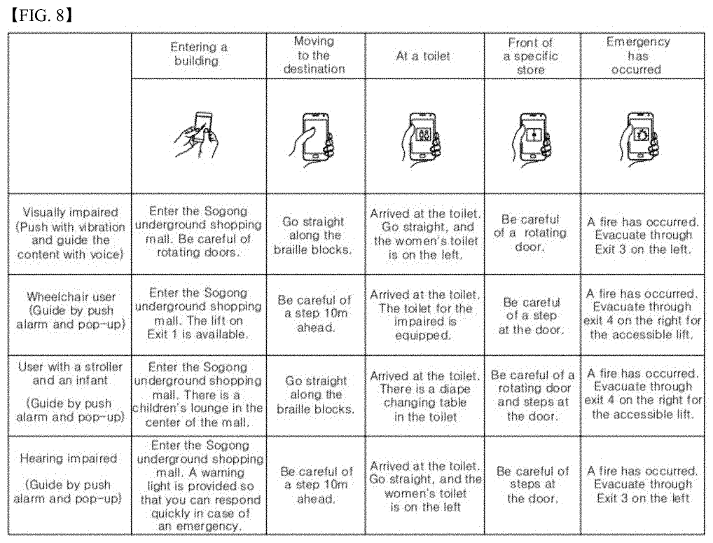

FIG. 8 shows one example in which different accessibility information is provided according to the socially disadvantaged type of the user when the user arrives at a predetermined destination according to one embodiment of the present invention. In the following description, unnecessary descriptions for embodiments redundant with those of FIGS. 1 to 7 will be omitted.

Meanwhile, in another embodiment of the present invention, when a predetermined destination information is inputted in the user terminal, a route from the current location received by a GPS module of the user terminal to the above-mentioned destination.

The above-mentioned route may be guided according to the socially disadvantaged type set in the user terminal, and the accessibility information on the corresponding spatial object may be provided every time when the user reaches a specific spatial object, including arriving at the above-mentioned destination, while guiding the route.

Referring to reference numeral 600 of FIG. 8 for a specific description, in the embodiment of 600, shows examples in which different accessibility information is provided according to the socially disadvantaged type, for example, entering a building, moving to the destination, at a toilet, front of a specific store, and upon emergency.

More specifically referring to the example of entering the building shown at 600, the user terminal of the visually impaired may be provided with the presence of a hinged door, the user terminal of the walking impaired using a wheelchair may be provided with the presence of a lift, the user terminal of the walking impaired with an infant may be provided with the presence of a baby lounge, and the user terminal of the hearing impaired may be provided with the provision of a warning light.

Meanwhile, at least one of vibration, push, voice and pop-up notification mechanisms may be used to notify the accessibility information, and the notification mechanisms may also be used differently for the socially disadvantaged types.

In other words, according to the present invention, the accessibility information essential for each socially disadvantaged type may be outputted and provided according to the socially disadvantaged type with respect to a predetermined spatial object by using the at least one notification mechanism, so that the enhanced customized geographic information service can be provided for the socially disadvantaged users.

Although the above embodiments have been described with reference to the limited embodiments and drawings, it will be understood by those skilled in the art that various changes and modifications may be made from the above-mentioned description.

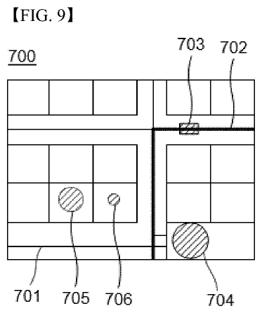

FIG. 9 shows one example for explaining a flow in which control information is managed according to one embodiment of the present invention. In the following description, unnecessary descriptions for embodiments redundant with those of FIGS. 1 to 8 will be omitted.

Reference numeral 700 of FIG. 9 is an example of a screen on which control information is managed. The screen is a screen outputted to a management terminal or the like of the universal map server, and may be outputted to an interface accessible by an administrator account for managing the universal map server.

In addition, it may be understood that the screen may be defined and outputted for each socially disadvantaged type, and different information may be outputted in the above-described interface when the socially disadvantaged type is switched.

Referring to FIG. 9, for a specific map, information 702, 704, 705, 706 about the time the user stayed in a specific place is outputted by using location information variation values of the user terminal, so that absolute and relative values about times and places in which a large number of users stay may be checked, and thus information on preferred places of stay may be determined.

In addition, routes 701 and 703 of users may be determined using the location variation values of the user terminals, and superimposed with respect to a plurality of users to divide the routes into the route 703 on which a majority of users move and the route 701 on which a minority of users move, thereby generating preferred route information.

In other words, according to the present invention, both of the preferred and non-preferred routes are determined according to the socially disadvantaged type, so that more sophisticatedly extracted geographic information can be provided to the socially disadvantaged users.

Although the above embodiments have been described with reference to the limited embodiments and drawings, it will be understood by those skilled in the art that various changes and modifications may be made from the above-mentioned description.

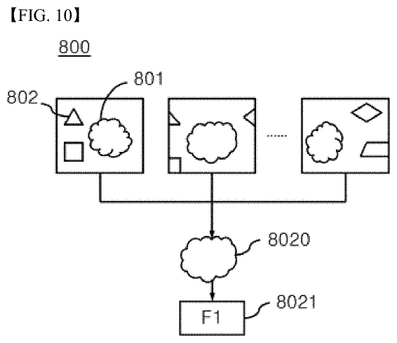

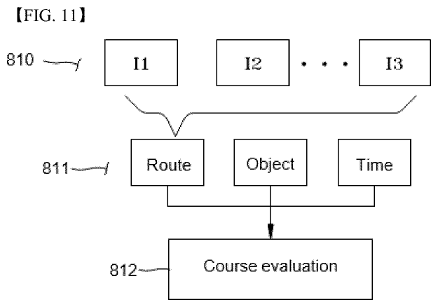

Meanwhile, FIGS. 10 and 11 show examples in which images photographed by a user terminal are utilized according to one embodiment of the present invention. In the following description, unnecessary descriptions for embodiments redundant with those of FIGS. 1 to 9 will be omitted.

Referring to FIG. 10 first, a photographed image 800 includes a plurality of objects 801 and 802, and the photographed image 800 refers to an image photographed by one user.

When time for the specific object 801 included in the photographed image 800 is longer compared to other objects, this may be determined that the user is looking at the object carefully and prefers the object.

In this case, the user terminal may define an image of only the object in the above example, that is, an image 8020 obtained by separating only the image of the object from the photographed image, as a first image 8021, and control information including the first image may be transmitted to the universal map server.

In the above-described embodiment, the time and location information at which the first image 8021 is measured may be combined with the control information.

Meanwhile, referring to FIG. 11, a plurality of photographed images 810 are measured, and information different from that of FIG. 10 is generated according to the performance of the function mentioned in the description of FIG. 10.

In other words, when information on the image of the photographed image includes GIS information, that is, location information of the user terminal and time information measured by a timer, the user's route and time (including moving time, time point, stay time, and the like) may be identified by using the image, location information and time information as mentioned in the first embodiment, and information on the object at which the user is looking on the corresponding route may be used as control information 811.

In this case, the universal map server, based on the corresponding information, may check course evaluation information 812, that is, information on usage rate and preference of users for facilities and the like according to the provided socially disadvantaged type, so as to determine specific facilities or the like mainly used or interestedly viewed by the user.

Meanwhile, as another embodiment using the above-described control information according to the present invention, the commercial district analysis may be performed by using the number of floating population and the stay time of the socially disadvantaged users.

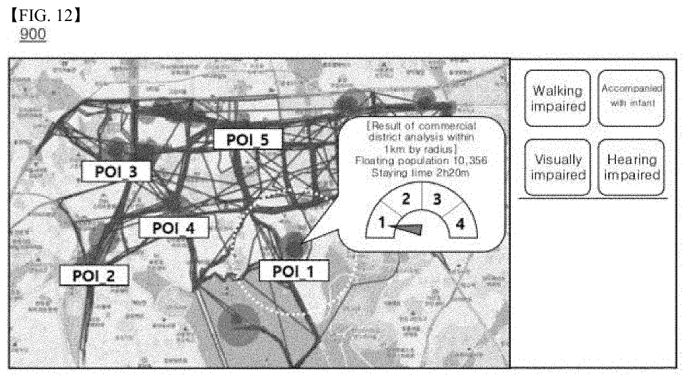

Screen 900 shown in FIG. 12 shows an embodiment in which the result of performing the commercial district analysis is provided to the user terminal. In the following description, unnecessary descriptions for embodiments redundant with those of FIGS. 1 to 11 will be omitted.

As described above, the information on floating population number and stay time of the socially disadvantaged users for POI in one area may be extracted using the control information, so that a POI-specific commercial district analysis may be performed.

As shown in screen 900 of FIG. 12, the commercial district analysis may be performed by analyzing the number of floating population and the stay time in a preset distance radius based on the location of the POI using the control information, so that a commercial district grade for each POI may be set and visualized on the geographic information outputted to the user terminal.

In other words, according to the present invention, the POI-specific commercial district analysis results may be visualized and provided to the geographic information provided to the user terminal, so that a mechanism for determining a congested district of facilities or devices conveniently used according to the socially disadvantaged type can be provided together.

Different commercial district analysis results may be derived for each socially disadvantaged type, and accordingly, it may be understood that the commercial district analysis results according to the socially disadvantaged type may be outputted differently depending on the characteristics of each socially disadvantaged type.

Meanwhile, as another embodiment of the present invention, the above-described user terminal as a user-wearable device may interwork with an EEG analyzer capable of measuring the user's electroencephalogram, and may collect EEG information according to the user's location and time point in conjunction with a sensor capable of collecting GIS and a timer that are mounted on the user terminal.

The universal map server may generate emotional information for each situation including information about the user's emotions at the user's location and time point by using the user's location, time point, and EEG information collected from the user terminal, generate preference information about facilities for each socially disadvantaged type from users' situational emotional information collected from a plurality of user terminals, and store the preference information in the database.

Referring to FIG. 13 for a more detailed description, FIG. 13 shows one example for explaining a flow in which emotional information for each situation is utilized according to one embodiment of the present invention. In the following description, unnecessary descriptions for embodiments redundant with those of FIGS. 1 to 12 will be omitted.

Referring to screen 1000 of FIG. 13, when EEG information (emotional information) generated by an EEG analyzer 1010 is transmitted to a user terminal 30, the user terminal 30 combines the EEG information with the information on place and time collected from the sensor of the user terminal 30 and transmits the combined information collection to a universal map server 20.

The universal map server 20 may generate and manage emotion information for each situation by using the received information to collect the emotion information from a plurality of user terminals 30, and generate information on a preferred facility 1030 for each socially disadvantaged type by using the collected data and store the generated information in a database 1020.

For the emotion information for each situation, preference information for each socially disadvantaged type is used so that facilities within a second threshold sequence (for example, 3) preset in order of high preference are classified as support information, and facilities within a third threshold sequence (for example, 4) preset in order of low preference are classified as obstacle information, and the classified information may be transmitted to a terminal of the administrative agency.

In other words, according to the present invention, it may be understood that information on preferred facilities with high emotional preference or information on non-preferred facilities with low emotional preference based on electroencephalogram collected from the user terminal 30 may be provided as the geographic information. Accordingly, the geographical information services expanded to various categories can be provided to the socially disadvantaged users.

On the other hand, as another embodiment of the present invention, the user terminal serving as a user-wearable device (for example, a smart watch) may interwork with a heart rate sensor capable of measuring the user's heart rates to collect heart rate information according to the user's location and time point in conjunction with a sensor capable of collecting GIS and a timer that are mounted on the user terminal.

The universal map server may control the user's heart rate information at the user's location and time point by using the user's location, time point, and heart rate information collected from the user terminal.

Referring to FIG. 14 for a more detailed description, screen 1100 of FIG. 14 shows an example of outputting an interface for controlling heart rate information collected from a plurality of user terminals.

In other words, it may be understood that, as shown in screen 1100 of FIG. 14, the manager terminal having an allowed access to the interface for controlling the heart rate information may control the heart rate information according to the socially disadvantaged type, or control the heart rate information by searching for the ID of a specific user.

Meanwhile, the universal map server may function to define emergency situations by categorizing the user's urgency according to measured heart rate values, and take different actions according to the urgency.

Specifically, the urgency of the user's heart rate information may be classified as “caution” when the user's heart rate is less than or equal to 50 or greater than equal to 120, and the urgency may be classified as “dangerous” when the user's heart rate is less than or equal to 40. When the urgency is “caution”, the action of providing location information and status notifications to the user terminal or control agency terminal is performed. When the urgency is “dangerous”, the action of sending a rescue request message to a previously set guardian contact or a rescue agency terminal such as 911 call may be performed while simultaneously providing the location information and the status notifications to the user terminal and the control agency terminal.

According to the present invention, it may be desirable that, when an increased state of the heart rate continues for 5 minutes or more based on the heart rate information collected from the user terminal, the user may be determined as exercising, thereby preventing notification information to be sent to the control agency from being generated, so as to improve the accuracy of determination on the user's emergency.

Although the above embodiments have been described with reference to the limited embodiments and drawings, it will be understood by those skilled in the art that various changes and modifications may be made from the above-mentioned description.

In addition, the aforementioned embodiments may be performed independently of each other, however, may be combined with each other to perform new and various functions as described above, and various changes and modifications may be made from the above-mentioned description.

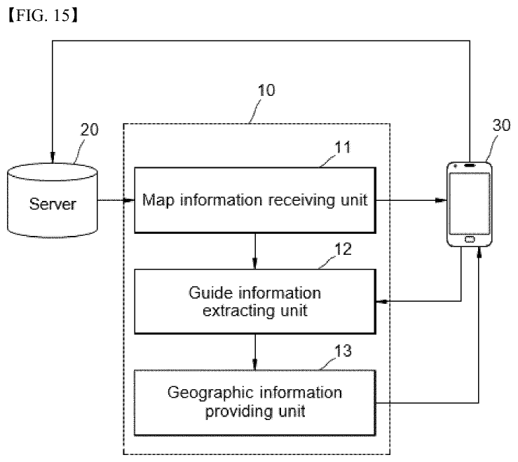

FIG. 15 shows a configuration diagram of a device 10 for providing geographic information and analysis information for each user using a universal map according to one embodiment of the present invention. In the following description, unnecessary descriptions for embodiments redundant with those of FIGS. 1 to 14 will be omitted.

As shown in FIG. 15, the device 10 for providing the geographic information and the analysis information for each user using the universal map may include a map information receiving unit 11, a guide information extracting unit 12, and a geographic information providing unit 13.

The above-mentioned map information receiving unit 11 may function to request authentication to a universal map server 20 storing map information including at least one of a map image and guide information for each socially disadvantaged type by transmitting user authentication information except for socially disadvantaged type information of a user among user account information requesting the authentication, and receive map information from the universal map server 20 when the authentication is completed.

In other words, it may be understood that the above-mentioned map information receiving unit 11 functions to allow the user terminal 30 to receive map information including guide information for all socially disadvantaged types. As a result, it may be preferable to understand that all functions performed in step S10 of FIG. 1 can be performed.

Meanwhile, the above-described guide information extracting unit 12, as guide information for providing the geographic information on the map image to the user terminal 30 from the received map information, functions to extract guide information corresponding to the socially disadvantaged type information of the user among the user account information from a data model database storing data models for each socially disadvantaged type.

In other words, it may be understood that the above-described guide information extracting unit 12 may function to extract only the guide information corresponding to the user's socially disadvantaged type from the map information including all previously received guide information for each socially disadvantaged type so as to allow the extracted guide information to be outputted to an output mechanism of the user terminal 30.

As a result, it may be understood that the above-described guide information extracting unit 12 can perform all functions performed in step S20 of FIG. 1.

On the other hand, the above-described geographical information providing unit 13 functions to provide customized geographic information according to the user's socially disadvantaged type information by outputting and providing geographic information reflecting attribute information of spatial objects existing on the map image based on the guide information extracted to the user terminal 30.

At this point, the above-described geographical information providing unit 13 may visualize and output different surrounding POI information according to the user's socially disadvantaged type on the map image activated in the output mechanism of the user terminal 30, and accordingly, the geographic information providing mechanism with maximized recognition convenience can be provided to the user belonging to the socially disadvantaged class.

In addition, the above-described geographical information providing unit 13 may be understood that the geographical information output mechanism may be set differently from each other based on the socially disadvantaged type of the user. As an example, it may be understood that the geographic information is guided by using a TTS technology for the visually impaired, by using a sign language video for the hearing impaired, and based on the POI list for the walking impaired.

In addition, the above-described geographical information providing unit 13 provides information on the support factors and the obstacle factors to the user terminal 30, including accessibility information on support factors and obstacle factors included in attribute information of spatial objects, so that convenience in mobility can be maximized.

In addition, it may be understood that the above-described geographical information providing unit 13 may also function to provide the use difficulty information included in the geographic information and determine the deviation from the route, and re-output the geographic information reflecting the bypass instruction.

In other words, it may be understood that the above-described geographical information providing unit 13 can perform all functions performed in step S20 of FIG. 1.

Thus, according to the device 10 for providing the geographic information and the analysis information for each user using the universal map of the present invention, the map information is received after only the authentication information except for the socially disadvantaged type information of the user terminal 30 is provided to the universal map server 20, so that personal information specifying the user can be prevented from being leaked to the network.

In addition, a universal map, which stores map information including at least one of a map image and guide information for each socially disadvantaged type, is received, and only the guide information according to the socially disadvantaged type of the user is extracted from the user terminal 30 and outputted to an output mechanism of the user terminal 30, geographic information, which is mainly focused on attribute information of spatial objects including the support factor information and the obstacle factor information, can be provided as geographic information according to the socially disadvantaged type of the user.

In addition, the output mechanism for providing the geographic information is also customized according to the socially disadvantaged type to which the user belongs, so that desired geographic information can be easily obtained without any particular difficulty, and the weight of accessibility information including at least one of support factor information and obstacle factor information is set according to the socially disadvantaged type information and the attribute information of the spatial object and the sequence to be preferentially outputted to the user terminal 30 is determined according to the set weight, thereby ensuring the simplicity of an output system of the geographic information, so that the convenience in use can be maximized.

In addition, a feature value of the accessibility information in which the bypass instruction is inputted is analyzed among the provided geographic information to provide geographic information excluding accessibility information having a feature value greater than or equal to a threshold similarity when following geographic information is provided, so that a more enhanced customized geographic information service can be provided according to using patterns of the user.

Although the above embodiments have been described with reference to the limited embodiments and drawings, it will be understood by those skilled in the art that various changes and modifications may be made from the above-mentioned description.

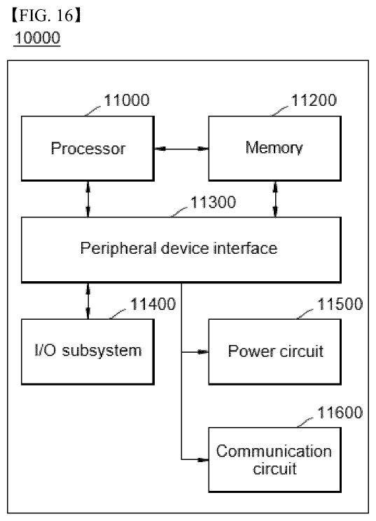

On the other hand, FIG. 16 shows one example of an internal configuration of a computing device according to one embodiment of the present invention. In the following description, unnecessary descriptions for embodiments redundant with those of FIGS. 1 to 15 will be omitted.

As shown in FIG. 16, the computing device 10000 may at least include at least one processor 11100, a memory 11200, a peripheral device interface 11300, an input/output subsystem (I/O subsystem) 11400, a power circuit 11500, and a communication circuit 11600. The computing device 10000 may correspond to a user terminal A connected to a tactile interface device or correspond to the above-mentioned computing device B.

The memory 11200 may include, for example, a high-speed random access memory, a magnetic disk, an SRAM, a DRAM, a ROM, a flash memory, or a non-volatile memory. The memory 11200 may include a software module, an instruction set, or other various data necessary for the operation of the computing device 10000.

The access to the memory 11200 from other components of the processor 11100 or the peripheral interface 11300, may be controlled by the processor 11100.

The Peripheral interface 11300 may combine an input and/or output peripheral device of the computing device 10000 to the processor 11100 and the memory 11200. The processor 11100 executes the software module or the instruction set stored in memory 11200, thereby performing various functions for the computing device 10000 and processing data.

The I/O subsystem 11400 may combine various input/output peripheral devices to the peripheral interface 11300. For example, the input/output subsystem 11400 may include a controller for combining the peripheral device such as monitor, keyboard, mouse, printer, or a touch screen or sensor, if needed, to the peripheral interface 11300. According to another aspect, the input/output peripheral devices may be combined to the peripheral interface 11300 without passing through the I/O subsystem 11400.

The power circuit 11500 may provide power to all or a portion of the components of the terminal. For example, the power circuit 11500 may include a power failure detection circuit, a power converter or inverter, a power status indicator, a power failure detection circuit, a power converter or inverter, a power status indicator, or any other components for generating, managing, and distributing the power.

The communication circuit 11600 uses at least one external port, thereby enabling communication with other computing devices.

Alternatively, as described above, the communication circuit 11600 may transmit and receive an RF signal, also known as an electromagnetic signal, including RF circuitry as needed, thereby enabling communication with other computing devices.

The above embodiment of FIG. 16 is merely an example of the computing device 10000, and the computing device 11000 may have a configuration or arrangement, in which some components shown in FIG. 16 are omitted, additional components not shown in FIG. 16 are further provided, or at least two components are combined. For example, a computing device for a communication terminal in a mobile environment may further include a touch screen, a sensor or the like, in addition to the components shown in FIG. 16, and the communication circuit 1160 may include a circuit for RF communication of various communication schemes (such as WiFi, 3G, LTE, Bluetooth, NFC, and Zigbee). The components that may be included in the computing device 10000 may be implemented by hardware, software, or a combination of both hardware and software which include at least one integrated circuit specialized in a signal processing or an application.

The methods according to the embodiments of the present invention may be implemented in the form of program instructions to be executed through various computing devices, so as to be recorded in a computer-readable recording medium. In particular, a program according to the embodiment of the present invention may be configured as a PC-based program or an application dedicated to a mobile terminal. The application to which the present invention is applied may be installed in a user terminal through a file provided by a file distribution system. For example, the file distribution system may include a file transmission unit (not shown) for transmitting the file according to a request of the user terminal.

The above-mentioned device may be implemented by hardware components, software components, and/or a combination of the hardware components and the software components. For example, the devices and components described in the embodiments may be implemented by using at least one general purpose computer or special purpose computer, for example, a processor, a controller, an arithmetic logic unit (ALU), a digital signal processor, a microcomputer, a field programmable gate array (FPGA), a programmable logic unit (PLU), a microprocessor, or any other device capable of executing and responding to instructions. The processing device may execute an operating system (OS) and at least one software application executed on the operating system. In addition, the processing device may access, store, manipulate, process, and generate data in response to the execution of the software. For the further understanding, some cases may have described that one processing device is used, however, it will be appreciated by those skilled in the art that the processing device may include a plurality of processing elements and/or a plurality of types of processing elements. For example, the processing device may include a plurality of processors or one processor and one controller. In addition, other processing configurations, such as a parallel processor, are also possible.

The software may include computer program, code, instruction, or at least one combination thereof, and may configure the processing device to operate as desired, or may instruct the processing device independently or collectively.

In order to be interpreted by the processor or to provide instructions or data to the processor, the software and/or data may be permanently or temporarily embodied in any type of machine, component, physical device, virtual equipment, and computer storage medium or device. The software may be distributed over computing devices connected to networks, so as to be stored or executed in a distributed manner. The software and data may be stored in at least one computer-readable recording media.

The method according to the embodiment may be implemented in the form of program instructions to be executed through various computing mechanisms, so as to be recorded in a computer-readable recording medium. The computer-readable recording medium may include program instructions, data files, data structures, and the like, independently or in combination thereof. The program instructions recorded on the media may be specially designed and configured for the embodiment, or may be known to those skilled in the art of computer software so as to be used. An example of the computer-readable medium includes a magnetic media such as a hard disk, a floppy disk and a magnetic tape, an optical media such as a CD-ROM and a DVD, a magneto-optical media such as a floptical disk, and a hardware device specially configured to store and execute a program instruction such as ROM, RAM, and flash memory. An example of the program instruction includes a high-level language code to be executed by a computer using an interpreter or the like as well as a machine code generated by a compiler. The above hardware device may be configured to operate as at least one software module to perform the operations of the embodiments, and vise versa.

Although the above embodiments have been described with reference to the limited embodiments and drawings, it will be understood by those skilled in the art that various changes and modifications may be made from the above-mentioned description. For example, even though the described descriptions are performed in an order different from the described manner, and/or the described components such as system, structure, device, and circuit are coupled or combined in a form different from the described manner, or replaced or substituted by other components or equivalents, appropriate results may be achieved. Therefore, other implementations, other embodiments, and equivalents to the claims are also within the scope of the following claims.

What is claimed is:

1. A method implemented in a user terminal including at least one processor and at least one memory for storing instructions executable by the processor to provide geographic information and analysis information for each user using a universal map, the method comprising:
a map information receiving step of requesting authentication to a universal map server storing map information including at least one of a map image and guide information for each socially disadvantaged type by transmitting user authentication information except for socially disadvantaged type information of a user among user account information requesting the authentication, so as to receive map information from the universal map server when the authentication is completed;
a guide information extraction step of extracting guide information, as guide information for providing the geographic information on the map image to the user terminal from the received map information, corresponding to the socially disadvantaged type information of the user among the user account information from a data model database storing data models for each socially disadvantaged type; and
a geographic information providing step of providing geographic information customized according to the socially disadvantaged type information of the user, by outputting and providing geographic information reflecting attribute information of spatial objects existing on the map image to the user terminal based on the extracted guide information.

2. The method of claim 1, wherein the geographical information providing step includes visually outputting and providing different surrounding POI information, as the geographic information, according to the socially disadvantaged type of the user on a map image activated in the user terminal.

3. The method of claim 1, wherein the geographical information providing step is performed through an output mechanism for providing the geographic information according to the socially disadvantaged type of the user, in which the geographic information is converted and outputted from text information into voice signals by using a text-to-speech (TTS) technology for a visually impaired, the geographic information is outputted as a sign language image for a hearing impaired, and the geographic information is outputted as a point of interest (POI) list for a walking impaired.

4. The method of claim 1, wherein the attribute information of the spatial object includes accessibility information for support factor information and obstacle factor information, and the support factor information and the obstacle factor information are classified based on different classification criteria according to the socially disadvantaged type.

5. The method of claim 4, wherein, in the geographical information providing step, a weight is set to accessibility information including at least one of the support factor information and the obstacle factor information according to socially disadvantaged type information of the user and attribute information of the spatial object, so as to determine a sequence of accessibility information to be outputted preferentially to the user terminal when the geographic information providing step is performed, wherein
the accessibility information is outputted to the user terminal in a form of an overlay with at least one screen displaying the accessibility information, in which transparency of the screen is controlled according to the weight set to the accessibility information, so that a screen set as having a highest weight is outputted most opaquely.

6. The method of claim 4, wherein the geographical information providing step includes
providing the user terminal with preview information on accessibility information including at least one of the support factor information and the obstacle factor information, wherein
geographic information for bypassing one accessibility information in which the bypass instruction is inputted is re-outputted and provided when the user terminal determines that a bypass instruction is inputted with respect to the one accessibility information included in the provided preview information, and a feature value of the accessibility information in which the bypass instruction is inputted is analyzed, so as to provide geographic information automatically converted as geographic information having a route that bypasses accessibility information having a feature value greater than or equal to a preset similarity, when following geographic information is provided.

7. The method of claim 4, wherein the geographical information providing step includes predicting use difficulty in which an item including at least one of numbers and density of the support factor information and the obstacle factor information included in the provided geographic information is reflected, so as to further provide predicted use difficulty information on the geographic information to the user terminal.

8. The method of claim 1, wherein the user terminal collects information on the time the user stayed in a specific area and route information for a preset period of the user, as control information, by using stay time information for each location information on an area in which the user is located, and variation value information on the location information, measured from a sensor capable of collecting geographic information system (GIS) and a timer that are mounted on the user terminal, and
the universal map server collects preferred stay place information derived based on stay time information for one area among the control information collected by a plurality of user terminals according to the socially disadvantaged type so as to generate and store preferred staying place information for each socially disadvantaged type in a database, and collects preferred route information derived by the matching rate of route information during a preset period for each socially disadvantaged type, among the location information collected by the user terminals so as to generate and store preferred route information for each socially disadvantaged type in the database.

9. The method of claim 8, wherein the universal map server performs commercial district analysis by extracting floating population number and stay time information for a point of interest (POI) in one area from the control information collected by the terminals, and classifies a commercial district grade for each POI according to the commercial district analysis result to store the classified commercial district grade in the database, so that the commercial district grade for each POI is visually outputted and provided on the geographic information provided to the user terminal when the geographic information providing step is performed.

10. The method of claim 1, wherein the user terminal collects, as control information, a first image, which is a photographed image corresponding to a time for which an object included in a field of view (FOV) is identically maintained exceeds a preset first threshold time, among photographed images of the user's FOV photographed by a photographing device of the user terminal, and
the universal map server identifies an object included in the first images collected from the user terminals through a comparison process with previously stored images of facilities to derive facility information corresponding to the objects, generates preferred facility information with respect to the facility information within a preset first threshold order in the order of the number of derived times among the derived facility information, and then classifies the generated preferred facility information based on the socially disadvantaged type, so as to generate and store information on preferred facilities according to socially disadvantaged type in the database.

11. The method of claim 1, wherein the user terminal serving as a user-wearable device interworks with an electroencephalogram (EEG) analyzer capable of measuring the user's electroencephalogram, and collects EEG information according to the user's location and time point in conjunction with a sensor capable of collecting geographic information system (GIS) and a timer that are mounted on the user terminal, and
the universal map server generates emotional information for each situation including information about the user's emotions at the user's location and time point by using the user's location, time point, and EEG information collected from the user terminal, generates preference information about facilities for each socially disadvantaged type from users' situational emotional information collected from a plurality of user terminals, and stores the generated preference information in the database.

12. The method of claim 1, wherein the user terminal serving as a user-wearable device interworks with a heart rate sensor capable of measuring the user's heart rates, and collects heart rate information according to the user's location and time point in conjunction with a sensor capable of collecting geographic information system (GIS) and a timer that are mounted on the user terminal, and
the universal map server controls the user's heart rate information at the user's location and time point by using user's location, time point, and heart rate information collected from the user terminal, and classifies a degree of urgency of the user according to a measured heart rate value when the user's heart rate information is determined as being out of a normal heart rate range, so as to transmit an emergency status notification of the user according to the classified degree of urgency to a terminal of a control agency.

13. A device implemented by a user terminal including at least one processor and at least one memory for storing instructions executable by the processor to provide geographic information and analysis information for each user using a universal map, the device comprising:
a map information receiving unit configured to request authentication to the universal map server storing map information including at least one of a map image and guide information for each socially disadvantaged type by transmitting user authentication information except for socially disadvantaged type information of a user among user account information requesting the authentication, and receive map information from the universal map server when the authentication is completed;
a guide information extraction unit for extracting guide information, as guide information for providing geographic information on the map image to the user terminal from the received map information, corresponding to the socially disadvantaged type information of the user among the user account information from a data model database storing data models for each socially disadvantaged type; and
a geographic information providing unit for providing geographic information customized according to the socially disadvantaged type information of the user, by outputting and providing geographic information reflecting attribute information of spatial objects existing on the map image to the user terminal based on the extracted guide information.

14. A computer-readable recording medium storing instructions for allowing a computing device to perform following steps comprising:
a map information receiving step of requesting authentication to a universal map server storing map information including at least one of a map image and guide information for each socially disadvantaged type by transmitting user authentication information except for socially disadvantaged type information of a user among user account information requesting the authentication, so as to receive map information from the universal map server when the authentication is completed;
a guide information extraction step of extracting guide information, as guide information for providing the geographic information on the map image to the user terminal from the received map information, corresponding to the socially disadvantaged type information of the user among the user account information from a data model database storing data models for each socially disadvantaged type; and
a geographic information providing step of providing geographic information customized according to the socially disadvantaged type information of the user, by outputting and providing geographic information reflecting attribute information of spatial objects existing on the map image to the user terminal based on the extracted guide information.
